(19) United States
(12) Patent Application Publication
(10) Pub. No.: US 2019/0070387 A1
(43) Pub. Date: Mar. 7, 2019

(54) SYSTEM AND METHODS FOR ACCESSING AND TREATING CEREBRAL VENOUS SINUS THROMBOSIS

(71) Applicant: Mayank GOYAL, Calgary (CA)

(72) Inventor: Mayank GOYAL, Calgary (CA)

(21) Appl. No.: 16/121,045

(22) Filed: Sep. 4, 2018

Related U.S. Application Data
(60) Provisional application No. 62/553,829, filed on Sep. 2, 2017.

(51) Int. Cl. A61M 25/00; A61B 17/3207; A61B 17/22; A61M 1/36

(52) U.S. Cl. CPC A61M 25/0023; A61M 25/005; A61B 17/320758; A61B 17/2202; A61B 2017/00991; A61M 2025/0006; A61B 2017/22079; A61M 2210/125; A61M 1/3659

(57) ABSTRACT

The invention relates to systems and methods for intracranial venous vessel access and a system for treatment of dural sinus thrombosis. In particular, a system including a co-axial combination of a steerable variable thickness microwire operatively supporting a tapered larger bore support and larger bore distal access catheter is described. Methods of advancing the intracranial access system through the venous vasculature are also described.

### FIELD OF THE INVENTION

The invention relates to systems and methods for intracranial venous vessel access as well as treatment of cerebral venous sinus. In particular, a system including a co-axial combination of a steerable variable thickness microwire operatively supporting a tapered larger bore support and larger bore distal access catheter is described. Methods of advancing the intracranial access system through the venous vasculature are also described.

### BACKGROUND OF THE INVENTION

Cerebral venous thrombosis (CVT) refers to occlusion of venous channels in the cranial cavity. These can generally be sub-characterized as dural venous sinus thrombosis (DVST), cortical vein thrombosis and deep cerebral vein thrombosis. These conditions often co-exist and the clinical presentation can be very similar and nonspecific. Furthermore, the diagnostic imaging features can be subtle.

DVST is the most common condition. DVST is most likely to affect any age women on the contraceptive pill. However, other risk factors include lifestyle factors, other hormonal factors, drugs, anatomical/trauma and disease conditions. As such, more specific risk factors can include smoking, pregnancy, puerperium, steroids, and hyperthyroidism, prothrombotic haematological conditions including protein S deficiency and polycythaemia, local factors including skull abnormalities, infections (especially mastoid sinus—dural sinus occlusive disease) and head injury (especially skull fractures that extends to a dural venous sinus) and systemic illness including dehydration, sepsis, malignancy and connective tissue disorders. DVST may also be from idiopathic causes.

Presentation is variable and can range from asymptomatic to coma and death. Typically, patients complain of headache, nausea, and vomiting. Neurological deficits are variable.

DVST can also affect the arachnoid granulation absorption of cerebrospinal fluid with the result that consequent cerebral swelling may occur. The subsequent venous hypertension can lead to edema and haemorrhage.

Once diagnosed, treatment can be challenging. Presently, systemic anticoagulation (e.g. heparin and warfarin) is still the first-line treatment for dural venous sinus thrombosis. Anticoagulation is usually required even in the setting of venous hemorrhage.

Interventional management includes microcatheter thrombolysis or thromboplasty. As discussed in greater detail below, microcatheter intervention can be challenging with currently available catheter systems. That is, due to the relative rarity of venous thrombosis (as compared to arterial ischemic stroke), physicians must use catheter systems designed and/or engineered for cerebral artery access.

However, the specifics of CVT and cranial venous anatomy both have particular features that can limit the effectiveness of arterial access catheters in the venous system.

Structurally, arterial access catheters are characterized by a maximum outside diameter (OD) of about 8 French (2.67 mm) (and usually much smaller). That is, due to the characteristics of arterial ischemic stroke including the progressive narrowing of the distal arterial vessels and the blood pressure of the arterial system, the maximum diameter of larger distal access catheters (DACs) is about 8 French. As a result, arterial recanalization procedures typically use various combinations of bi-, tri- and quadra-axial catheter systems to progressively gain access to more distal regions of the cerebral arteries where the thrombosis may be located.

For reference, Table 1 shows the comparison of French, metric and imperial units used in catheters.

| French | Circumference | Diameter | Diameter |
|---|---|---|---|
| Gauge | (mm) | (mm) | (inches) |
| 3 | 3.14 | 1 | 0.039 |
| 4 | 4.19 | 1.333 | 0.053 |
| 5 | 5.24 | 1.667 | 0.066 |
| 6 | 6.28 | 2 | 0.079 |
| 7 | 7.33 | 2.333 | 0.092 |
| 8 | 8.34 | 2.667 | 0.105 |
| 9 | 9.42 | 3 | 0.118 |
| 10 | 10.47 | 3.333 | 0.131 |
| 11 | 11.52 | 3.667 | 0.144 |
| 12 | 12.57 | 4 | 0.158 |
| 13 | 13.61 | 4.333 | 0.170 |
| 14 | 14.66 | 4.667 | 0.184 |
| 15 | 15.71 | 5 | 0.197 |
| 16 | 16.76 | 5.333 | 0.210 |
| 17 | 17.81 | 5.667 | 0.223 |
| 18 | 18.85 | 6 | 0.236 |
| 19 | 19.90 | 6.333 | 0.249 |
| 20 | 20.94 | 6.667 | 0.263 |
| 22 | 23.04 | 7.333 | 0.288 |
| 24 | 25.13 | 8 | 0.315 |
| 26 | 27.23 | 8.667 | 0.341 |
| 28 | 29.32 | 9.333 | 0.367 |
| 30 | 31.42 | 10 | 0.393 |
| 32 | 33.51 | 10.667 | 0.419 |
| 34 | 35.60 | 11.333 | 0.445 |

The design of arterial access catheters is specific to the arterial anatomy and various features and properties are incorporated into arterial catheters to enable their successful progress into the cranial vasculature to conduct various recanalization procedures.

In contrast, the cranial venous system has its own specific anatomical features that create unique problems to the navigation of catheters into the venous vasculature. Similarly, DVST is morphologically dissimilar to arterial thrombosis.

For example, the dural sinus has a larger diameter (around 1 cm) and DVST may present as a substantial compromise of this vessel along a length of the dural sinus as much as 20 cm. Moreover, the DVST thrombus will often present as a compromise in the lumen of the dural sinus (as opposed to complete blockage) along this distance. The residual lumen may be as little as 1 mm. Hence, placement of arterial access catheters within the dural sinus and aspiration of a clot using an 8 French (2.67 mm) distal access catheter may simply create a relatively small channel through the clot which does not significantly reduce the clot burden or alleviate the condition.

Accordingly, there has been a need for catheter systems specifically designed for and having the functionality to effectively be positioned within the cerebral venous vasculature to enable DVST treatment. In particular, there has been a need for catheter systems having the combination of size (i.e. outer and inner diameter) radial flexibility, radial compressive stiffness as well as outer and inner surface features to enable navigation of the catheter to positions in the venous vasculature to effect removal of a venous thrombus.

Key structures within the venous anatomy make it difficult to navigate larger diameter catheters into the brain. In a typical procedure, using available cerebral access catheters, access to the cerebral venous system is obtained through femoral veins. Catheters are advanced to the inferior vena cava, through the right atria to gain access to the superior vena cava (SVA). From the SVA, access to the internal jugular vein (IVA) is achieved, followed by access to the sigmoid sinus, transverse sinus, torcula and superior sagittal sinus. Alternatively, direct access to the internal jugular vein through percutaneous puncture in the neck is also feasible.

For a larger diameter catheter, navigation from the generally more pliant neck vessels (i.e. internal jugular vein) to the contained cerebral vessels (i.e. sigmoid sinus) is the most challenging. Both pliancy of the vessels and tortuosity can create issues in the navigation of larger diameter catheters.

### SUMMARY OF THE INVENTION

In accordance with the invention, there is provided a catheter system for accessing the cerebral venous system from the femoral vein or jugular vein comprising: an outer catheter (OC) having an OC distal end inner diameter (ID); an inner catheter (IC) having an IC distal tip end, an IC proximal end and an IC expanded section adjacent the IC distal tip end, the IC expanded section having a distal taper, a proximal taper and an outer diameter (OD), the OD substantially corresponding to the ID of the OC distal end ID, the expanded section for supporting the OC distal end during catheter placement within the cerebral venous vessels wherein the IC expanded section has a length sufficient to enable telescopic movement of the OC and IC relative to one another to enable successive advancement of the OC and IC within the cerebral venous vessels without causing separation of the OC distal end from the IC expanded section and wherein the IC expanded section and OC collectively have sufficient flexibility to progress through the cerebral venous vessels and sufficient stiffness to prevent separation of the OC distal end from the IC expanded section

In one embodiment, the IC is a bi-axial system where the IC distal tip end is an independently moveable microwire (MW) having a MW distal tip end and a MW proximal end telescopically engaged within the IC expanded section and wherein the MW and IC are independently moveable with respect to one another from outside the body.

In one embodiment, the OC is a bi-axial system including a first OC telescopically engaged with the IC, and where the first OC has a first OC distal end engageable with the IC expanded section and where the first OC includes a widening taper adjacent to and extending proximally from the first OC distal end; and, a second OC telescopically engaged with the first OC, and where the second OC has a second OC ID engageable with the first OC, the second OC having an ID enabling a recanalization procedure to be conducted through the second OC.

In various embodiments, the IC expanded section has a diameter of 13-22 French and/or the IC expanded section has a length of 4-12 cm. In one embodiment, the IC has an inner lumen to allow a steerable wire (0.014-0.035 inches).

In one embodiment, the OC catheter has a distal tip ID of 13-25 French and an OD of 15-27 French.

In another aspect, the invention describes a method of accessing the cerebral venous vessels of a patient with the catheter system describing herein, including the steps of: a) introducing the catheter system into the femoral vein of the patient; b) advancing an IC system and OC system from the femoral vein successively to the inferior vena cava, the right atria, the superior vena cava, the internal jugular vein, the sigmoid sinus and superior sagittal sinus where the IC and OC are telescopically moved with respect to one another and where the OC distal tip remains supported by the IC expanded section while the catheter system is entering the patient's cranial venous vessels. Typically, if it is a bi-axial system, the IC system includes a steerable wire of an appropriate size inside the IC to provide support and direction.

The method may also include the step of withdrawing the IC from the OC and utilizing the OC to conduct a thrombus retrieval treatment selected from any one of or a combination of recanalization by aspiration, drug delivery and ultrasonic ablation.

In one embodiment, the access to the cranial venous system is obtained through the internal jugular vein.

### BRIEF DESCRIPTION OF THE DRAWINGS

Various objects, features and advantages of the invention will be apparent from the following description of particular embodiments of the invention, as illustrated in the accompanying drawings. The drawings are not necessarily to scale, emphasis instead being placed upon illustrating the principles of various embodiments of the invention. Similar reference numerals indicate similar components.

FIG. 1 is a schematic sketch of brain vascular anatomy showing features of the venous vasculature and a representative DVST thrombus.

FIG. 2 is a sketch of a typical microwire, microcatheter and distal access catheter that may used for arterial recanalization procedures in accordance with the prior art and illustrating the problem of separation between internal microcatheters/wires and outer catheters that can bind or be problematic to advance through regions of higher tortuosity.

FIG. 2A is a sketch of the venous vasculature illustrating the problem of advancing a DAC over a microcatheter in a region of high tortuosity.

FIG. 3 is a sketch of a venous access system in accordance with one embodiment of the invention.

FIGS. 3A, 3B, 3C and 3D are side views of an assembled system (3A), distal access catheter (3B), expanded section (3C) and microwire (3D) in accordance with one embodiment of the invention.

FIG. 4 is a sketch of a quadra-axial system with two outer catheters, an inner expanded section and an inner microwire in accordance with one embodiment of the invention.

FIGS. 5A and 5B are schematic diagrams showing a distal access catheter containing a clot maceration device being advanced towards a clot where the clot is engulfed and macerated and clot particles are withdrawn via suction.

### DETAILED DESCRIPTION

With reference to the figures, systems and methods for accessing cerebral venous thrombi are described.

### Overview

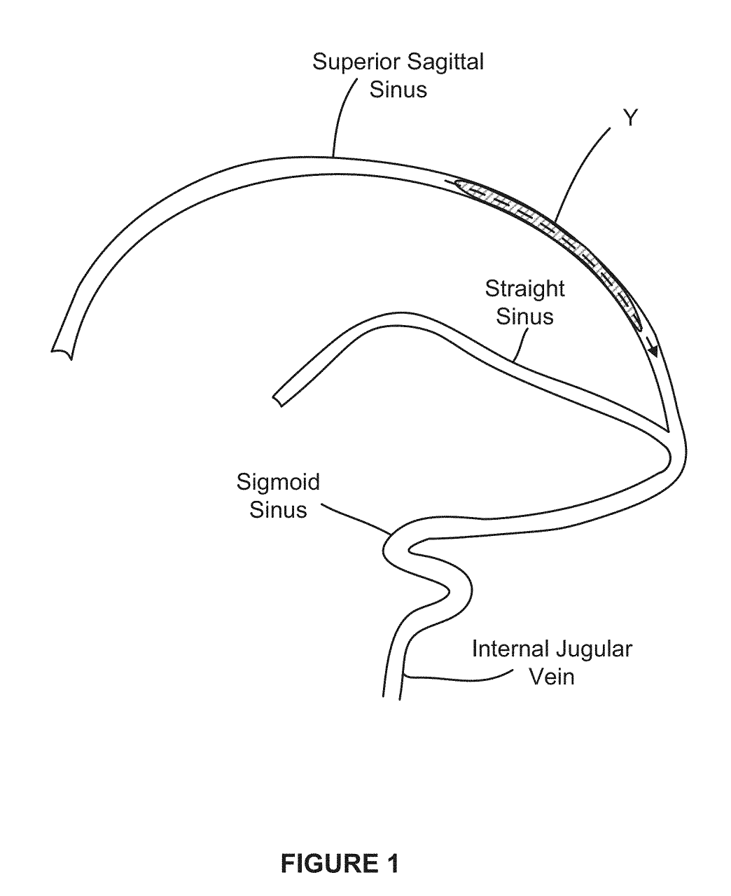

Cerebral Venous Thrombosis (CVT) is a rarer form of stroke occurring in the venous system. As shown schematically in FIG. 1, clots Y forming in the cerebral venous system (e.g. Superior Sagittal Sinus) can be manifested as a narrowing of the venous vessels to restrict blood flowing from the brain (dotted line). These clots may be both larger in diameter/length and volume as compared to cerebral clots. During recanalization, navigation of catheter systems to the superior sagittal sinus and/or transverse sinus and/or straight sinus (common sites of dural sinus thrombosis) generally progresses from the internal jugular vein, through the sigmoid sinus and into the superior sagittal sinus. In particular, navigation from the internal jugular vein to the sigmoid sinus can be difficult due to the tortuosity and pliancy of the vessels.

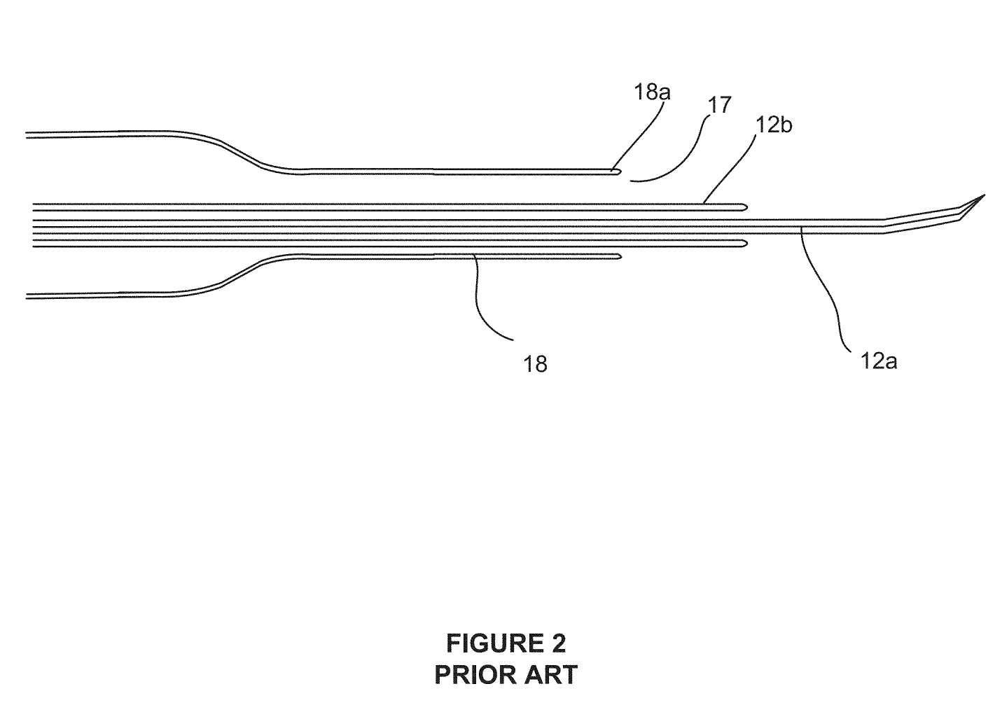

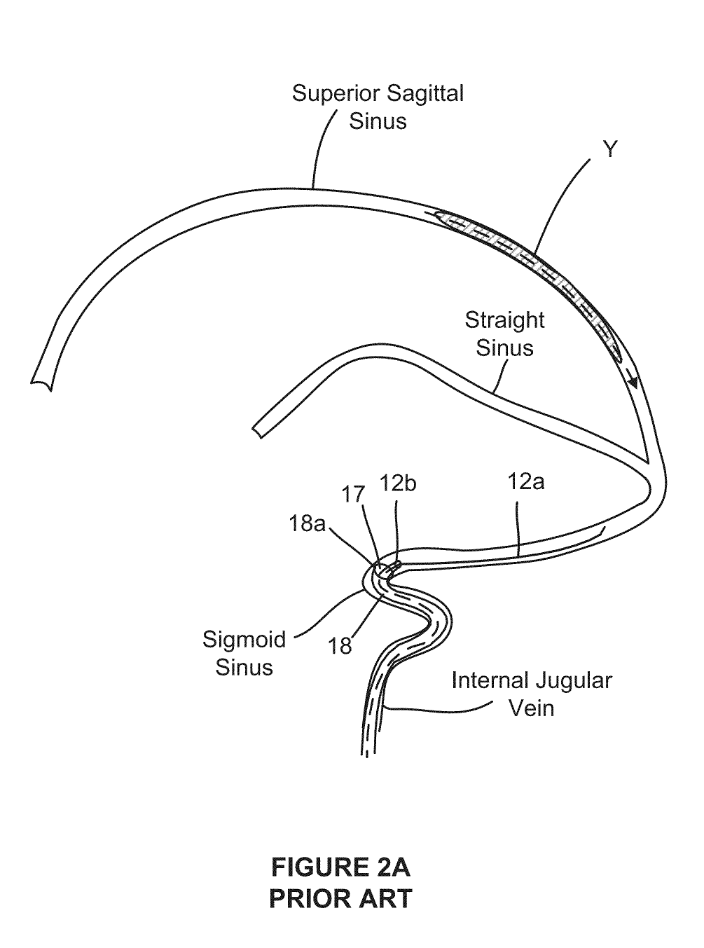

Moreover, the relative size of cerebral catheter systems compared to venous vessels can be problematic insomuch as the relatively smaller diameter and design of cerebral catheter systems can present problems as shown schematically in FIGS. 2 and 2A. Specifically, gaps 17 between the distal edge 18a of a larger catheter and smaller guide catheter can be difficult to get through regions of high tortuosity and/or regions of vessel pliancy.

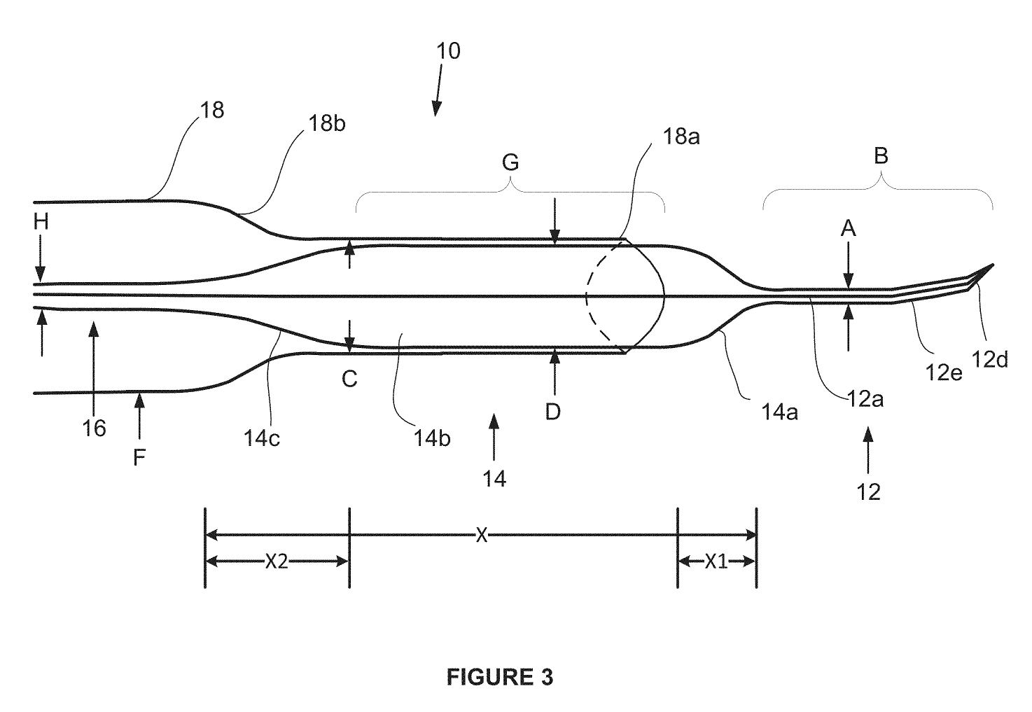

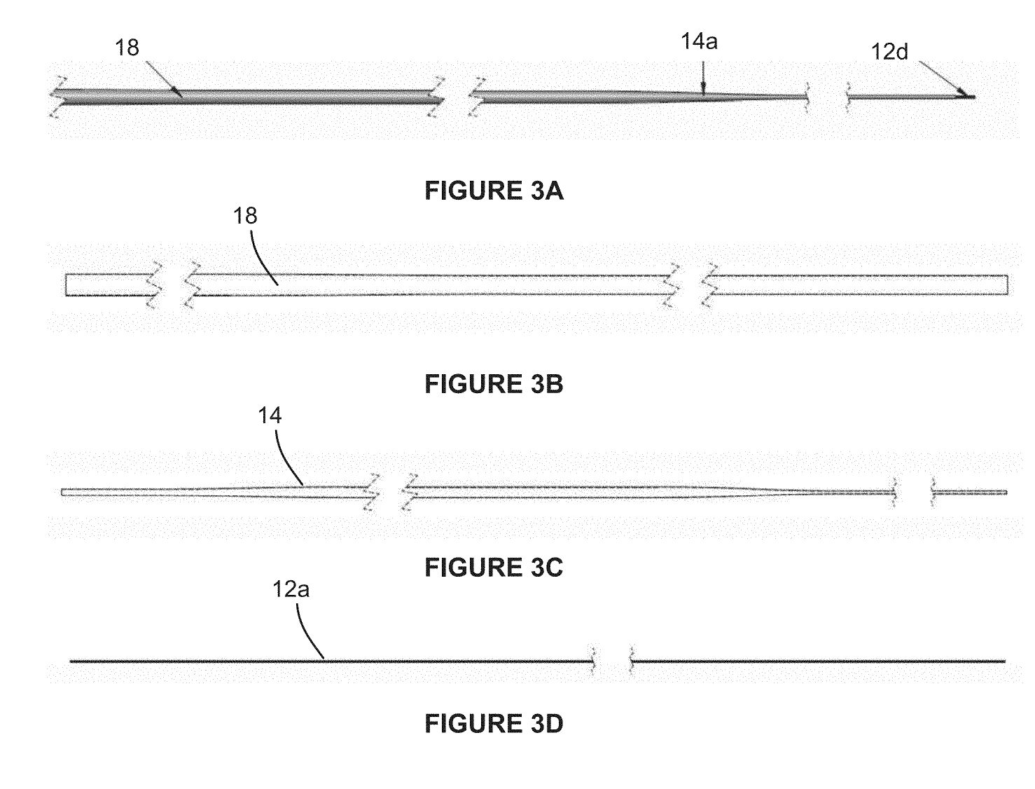

In a first embodiment, as shown in FIG. 3, the cerebral venous catheter system (CVCS) 10 comprises inner catheter (IC) system/components including a distal tip section 12, an expanded section 14 and a proximal support section 16 and an outer catheter (OC) system/components including at least one outer catheter such as a distal access catheter (DAC) 18 (also referred to herein as an aspiration catheter). As described in greater detail below, a distal portion of the IC components 14b is of a diameter that fits into and supports/engages with a distal end 18a of the OC components. Both the inner and outer components of the CVCS will typically have a total length of about 1.2 m and otherwise have sufficient length to be advanced from the femoral vein to the cerebral venous vessels. Shorter length catheters may be utilized if designed for jugular vein access. Generally, the differences in the lengths of each the inner and outer catheter components will be in the range of 10-20 cm. That is, the inner components will be around 10-20 cm longer than the outer components. Similarly, for tri- and quadra-axial systems, each component will have a length about 10-20 cm different from an adjacent component.

### Distal Tip Section 12

The distal tip section 12 is generally a thin wire 13 having a pre-formed or formable tip 12d that enables a physician to steer and advance the distal tip through the cerebral venous vasculature. The wire 13 will have an appropriate atraumatic coating 12e for intracranial use and will typically have an outer diameter A of 0.014-0.035 inch (1-2 F). The length B of the distal tip section will be in range of 10 cm. The inner catheter system will extend the entire length of the system and can be controlled by the physician from outside as a mono- or bi-, tri- or quadra-axial system as described below. It will typically be longer than the OC system.

### Expanded Section 14

The expanded section 14 provides a tapered transition from the narrower distal tip section 12 to the wider inner diameter C of a DAC 18. In the context of this description, a “taper” is generally referred to as a change in diameter from one section of the system to another. That is, a taper implies a narrowing of diameter from a region having one diameter to a region having a smaller diameter or vice versa. The purpose of the expanded section is to prevent separation of the distal edge 18a of the OC from the narrower distal tip section 12 and otherwise provide a smooth flexible surface at the junction of the inner and outer components as the distal region is being advanced and particularly as the distal region and DAC are being moved through areas of high vascular curvature and/or pliancy.

As shown, the expanded section 14 includes a distal tapered section 14a, a cylindrical central section 14b and a proximal tapered section 14c. The central section 14b will have an outer diameter D generally corresponding to the inner diameter C of the distal end of the DAC. As shown, the DAC may also include a DAC tapered section 18b that transitions the DAC from a narrower distal diameter C to a wider proximal diameter F.

Importantly, the inner components (namely the distal tip, expanded and proximal support sections) and the outer components (DAC) can move independently of each other. The inner components are steerable, namely a physician can by a combination of axial movement (i.e. distal or proximal pushing or pulling) and torque (i.e. twisting) can cause the distal tip to enter into desired vessels to gain access to a location where a recanalization procedure may be conducted. Generally, it would be expected that a skilled operator would alternately advance the inner components and subsequently advance the DAC over the inner components. The configuration would be maintained in a way that generally the distal end of the DAC would remain fixed in relation to the expanded section 14 of the inner components to allow for a smooth transition between the outer surfaces of both the inner components and DAC. That is, while the distal tip of the DAC is supported and the system is being pushed around a tight curve, the system does not create the gap 17 as shown in FIG. 2A which may otherwise prevent distal movement of the catheter system.

As can be seen, the central section 14 has a length G sufficiently long to enable this coaxial movement without causing the separation of the central section 14 from the distal inner diameter C of the DAC. In practice, the central section will have a length G of approximately 10 cm. The total length X of the expanded section 14 between the distal tip section 12 and proximal support section 16 will be about 12-17 cm. Thus, each of the tapered sections 14a (X₁) and 14c (X₂) will be about 2-6 cm long.

The outer diameter D of the central section 14b will be made to substantially match the inner diameter of the outer catheter. The outer catheter would range in size (outer diameter) of 5-9 mm (13-27 French) and would likely have an inner diameter of 11-25 French. The central section 14b is sufficiently strong in the radial direction while being bent to prevent separation of the DAC distal end 18a from the expanded section while moving around a tight curve. The central section 14b may have additional coating such as a hydrophilic coating to reduce friction.

The central section 14b and tapers 14a, 14c will preferably be a plastic/rubber section having sufficient flexibility to enable bending and movement through a tight curve and sufficient radial strength to prevent the tip separation as described above. In addition, it would preferably have a hydrophilic coating to reduce friction. There are generally two forms of the central section namely a) where the central guide wire is configured to the central section such that the two can only be moved together (i.e. mono-axial) and b) where the central section and proximal support section have a lumen allowing the guide wire to move independently of each other (i.e. bi-axial).

### Proximal Support Section 16

The proximal support section 16 will typically have an outer diameter H of about 5-8 French and have sufficient axial compressive strength to enable the distal tip section 12 to be pushed forward and sufficient torsional strength for turning and steering of the distal tip section 12.

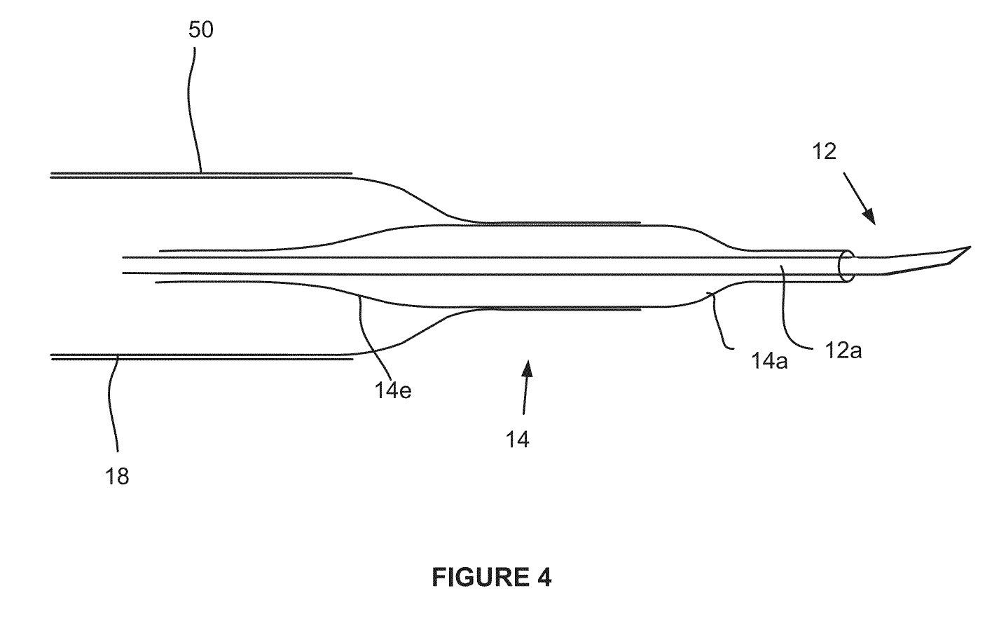

As shown in FIG. 4, in a bi-axial embodiment of the inner components, the distal tip region 12 and proximal support region 16 can be additionally coaxially moved relative to the expanded section 14. Thus, in this embodiment, the expanded region 14 forms a cover over the proximal support region having a tapered region proximal support section 14e that extends proximally and that enables the physician to independently slide these separate components relative to one another. In this embodiment expanded section 14 may be made of metal or polymers or a combination (using technologies used in making wires and microcatheters and DACs). As in the embodiment illustrated by FIG. 3, in this alternate embodiment, a DAC may be preloaded onto the inner components.

Further, the underlying wire 12a can be exchangeable so that if needed, once the distal access catheter is in place, inner components can be removed to conduct a recanalization procedure through the DAC.

FIG. 4 also shows an embodiment with multiple outer components, namely inner DAC 18 and outer DAC 50. In comparison to cerebral arteries where the maximum diameters are smaller, it may be desirable to use a tri- or quadra-axial system to gain access to the venous vessels using progressively wider DACs, where the inner DAC has tapered surfaces providing a transition from narrower to wider diameters. Generally, however, the outer DAC/aspiration catheter 50 does not need to be tapered due to the lower pressures on the venous side as compared to the arterial side. Thus, inner components may be bi-axial, meaning that the distal tip 12 is independently moveable relative to the expanded section 14 or mono-axial if they can only be moved together. Similarly, the outer components may be bi-axial if two co-axial DACs are utilized. In totality, the system will thus be bi-, tri- or quadra-axial.

FIG. 3A shows an assembly of a tri-axial IAS with FIGS. 3B, 3C and 3D showing each component including an outer DAC 18, expanded section 14 and inner wire 12a.

### Methods of Use

As described above, the system may be used to access an intracranial occlusion through a patient's venous vasculature. Generally, after the surgeon has gained access to the patient's vasculature at the femoral vein, the following general steps are followed:
a. Advance the inner and outer components from the femoral vein to the inferior vena cava.
b. Advance the inner and outer components through the right atria to gain access to the superior vena cava. During steps a and b, the physician will typically be advancing these components together sequentially using the inner wire (usually of 0.035 inches) to lead.
c. Gain access to the internal jugular vein. At this stage, the physician may advance the inner components ahead of the outer components, then hold the inner components and then advance the outer components. During this step, the physician will ensure that the inner and outer components are maintained at the appropriate spacing to ensure proper engagement of the expanded surfaces.
d. This process will be continued through the sigmoid sinus, etc. to the desired location to conduct the recanalization procedure.
e. Remove of the inner components and conduct recanalization procedure, typically by aspiration through the DAC. Other adjunctive recanalization technologies (such as thrombolytic pharmaceutical agents and/or mechanical agents such as ultrasonic liquefaction of the clot) could be introduced through the OC, once the OC is in position within the clot.
f. Variations may include a tri- or quadra-axial systems with successively larger DACs with appropriate adjustments in technique.
g. Aspiration may be conducted manually via a syringe or by a mechanical pump as known for arterial side clot removal.

### CVCS Advantages

Noted advantages of this solution are:
a. If the inner components are preloaded into the distal access catheter, preparation time for surgery will be reduced.
b. The system enables larger DACs specifically designed for the venous system to be safely positioned in the larger venous vessels.
c. Larger diameter catheter allows aspiration of clot across diameter of vessel which is not possible with arterial DACs.
d. Importantly, by aspirating the venous thrombus from the patient, natural repair/cleaning processes may be triggered to reduce the clot burden.
e. The system enables larger DACs to be utilized as a conduit for other treatment technologies by aspiration of the clot or introduction of other technologies or pharmaceutical agents within the clot to liquefy the clot.

In variations of the methodology, blood that is removed through the DAC may be returned to the body after cleaning to remove any blood clot debris. This step is desirable given the relatively larger volumes of blood being aspirated as a result of the larger diameter vessels and larger diameter catheters.

Units of measure used in this specification are consistent with the units used in the field of endovascular surgery. That is, both imperial and metric units are used where lengths are typically expressed in metric units while diameters can be expressed in imperial units.

Key features of the cerebral venous catheter system (CVCS) are shown in Table 2.

TABLE 2
Typical Dimensions and Properties

| | |
|---|---|
| Property | Measure |

Inner Catheter Components

| | |
|---|---|
| Overall Length | Longer (approximately 1.2 m) |
| Distal Tip Diameter | 2 F |
| Distal Tip Length | 10 cm |
| Option 1 | Fixed to Expanded Section (mono- |
| | axial) |
| Option 2 | Moveable relative to Expanded section |
| | (bi-axial) |
| Distal Taper Transition | 2-6 cm |
| Expanded Section length | 4-12 cm |
| Proximal taper transition | 2-6 cm |
| Expanded section Diameter | 11-25 F |

Outer Catheter Components

| | |
|---|---|
| Overall Length | Shorter (approximately 1.2 m) |
| Distal Tip Diameter | 13-27 F |
| Distal Taper Transition (if | 2-6 cm |
| utilized) | |
| Proximal Diameter | 13-27 F |
| Option 1 | Single DAC (may be fixed OD or taper |
| | from 13-27 F) (mono-axial system) |
| Option 2 | Second outer DAC with fixed OD (13-27 F) |
| | (bi-axial system) |

The inner and outer catheter systems will preferably include proximal and extracorporeal markings on their bodies that correspond to the relative end points of the IC expanded section (i.e. the IC expanded section length) to assist the physician in ensuring that during critical parts of the procedure, that the OC distal tip is not supported by the IC expanded section.

Corresponding changes in length for systems designed for intra-jugular access can be implemented.

### Clot Removal

As noted above, once a venous clot has been accessed by a DAC, clots may be removed by various techniques including aspiration techniques and/or various clot-busting techniques including chemical disruption via the use of pharmacological agents or mechanical disruption via the use of ultrasound or mechanical clot maceration.

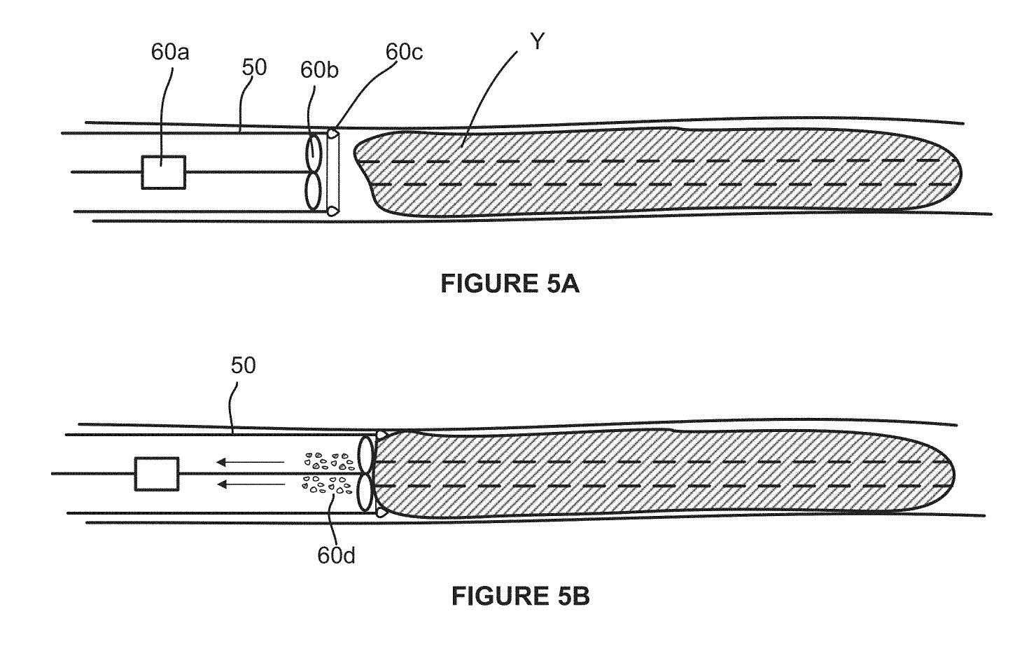

In the case of clot busting via mechanical disruption, ultrasound or clot maceration equipment 60 is deployed through a DAC 50 towards the distal end of the DAC when the DAC is adjacent a clot Y. As shown in FIG. 5A, a macerating motor 60a with connected impeller 60b is positioned proximally of the distal tip of the DAC. Preferably, the distal tip of the DAC has a lip 60c preventing inadvertent projection of the impeller beyond the distal end of the DAC. Preferably, the lip 60c is configured with a proximal edge having a profile substantially similar to the profile of the impeller that enables the impeller to lightly engage with the lip without stalling or jamming but that equally prevents the impeller from being extended past the lip.

The distal edge of the lip may also include an atraumatic profile that does not damage the vessel intima through which the DAC is being advanced but that encourages separation of the clot from the intima and into the DAC.

As shown in FIG. 5B, as the DAC is advanced, a portion of the clot Y may be engulfed by the DAC whereby it engages with the spinning impeller and macerated whereby clot particles 60d are withdrawn via suction.

Although the present invention has been described and illustrated with respect to preferred embodiments and preferred uses thereof, it is not to be so limited since modifications and changes can be made therein which are within the full, intended scope of the invention as understood by those skilled in the art. For example, dimensions provided herein are representative of normal ranges of sizes and can generally be considered to have tolerances of +/−10%. Additionally while the current system is specifically designed from the perspective of dural sinus thrombosis, it is expected that sites of venous thrombosis where in access is difficult due to tortuosity and large access catheters are needed for aspiration of the clot, similar systems with minor modifications could be made by those skilled in the art.

1. A catheter system for accessing the cerebral venous system from the femoral vein or jugular vein comprising:
an outer catheter (OC) having an OC distal end having an OC distal end inner diameter (ID);
an inner catheter (IC) having an IC distal tip end, an IC proximal end and an IC expanded section adjacent the IC distal tip end, the IC expanded section having a distal taper, a proximal taper and an outer diameter (OD), the OD substantially corresponding to the ID of the OC distal end ID, the expanded section for supporting the OC distal end during catheter placement within the cerebral venous vessels wherein the IC expanded section has a length sufficient to enable telescopic movement of the OC and IC relative to one another to enable successive advancement of the OC and IC within the cerebral venous vessels without causing separation of the OC distal end from the IC expanded section and wherein the IC expanded section and OC collectively have sufficient flexibility to progress through the cerebral venous vessels and sufficient stiffness to prevent separation of the OC distal end from the IC expanded section

2. The catheter system as in claim 1 where the IC is a bi-axial system where the IC distal tip end is an independently moveable microwire (MW) having a MW distal tip end and a MW proximal end telescopically engaged within the IC expanded section and wherein the MW and IC are independently moveable with respect to one another from outside the body.

3. The catheter system as in claim 1 where the OC is a bi-axial system including:
a first OC telescopically engaged with the IC, and where the first OC has a first OC distal end engageable with the IC expanded section and where the first OC includes a widening taper adjacent to and extending proximally from the first OC distal end; and,
a second OC telescopically engaged with the first OC, and where the second OC has a second OC ID engageable with the first OC, the second OC having an ID enabling a recanalization procedure to be conducted through the second OC.

4. The catheter system as in claim 1 where IC expanded section has a diameter of 11-25 French.

5. The catheter system as in claim 1 where the IC expanded section has a length of 4-12 cm.

6. The catheter system as in claim 1 where the OC catheter has an OC distal tip ID of 13-27 French.

7. The catheter system as in claim 1 where the OC catheter is a bi-axial system including an inner OC and an outer OC and wherein the inner OC has an OC distal tip ID of 13-16 French and has a proximally extending taper to an ID of 16-22 French.

8. The catheter system as in claim 1 where the IC and OC have lengths enabling femoral venous access to a patient.

9. The catheter system as in claim 1 where the IC and OC have lengths enabling jugular venous access to a patient.

10. A method of accessing the cerebral venous vessels of a patient with the catheter system of claim 1 comprising the steps of:
a) introducing the catheter system into the femoral vein of the patient;
b) advancing an IC and OC from the femoral vein successively to the inferior vena cava, the right atria, the superior vena cava, the internal jugular vein, the sigmoid sinus and superior sagittal sinus where the IC and OC are telescopically moved with respect to one another and where the OC distal tip remains supported by the IC expanded section while the catheter system is entering the patient's cranial venous vessels.

11. The method as in claim 10 further comprising the step of withdrawing the IC from the OC and utilizing the OC to conduct a thrombus retrieval treatment and recanalization by aspiration on its own or any combination of, drug delivery, mechanical maceration and ultrasonic ablation.

12. The method as in claim 11 further comprising the step of collecting and cleaning recovered blood and reintroducing recovered and cleaned blood back to the patient.

13. A distal access catheter (DAC) for placement within the cerebral venous vessels for venous thrombi removal, the DAC comprising:
a catheter body having an outer diameter generally corresponding to the diameter of a patient's superior sagittal sinus, and,
a distal tip on the catheter body defining an atraumatic lip, the atraumatic lip having a profile to prevent a macerating impeller deployable within the DAC from advancing beyond the distal tip.
